(19) United States
(12) Patent Application Publication
(10) Pub. No.: US 2024/0374527 A1
(43) Pub. Date: Nov. 14, 2024

(54) Pharmaceutical Composition Comprising Lumateperone

(71) Applicant: Alkem Laboratories Limited, Mumbai, Maharashtra (IN)

(72) Inventors: Madhusudan Hanchate, Dombivli East (IN); Vaibhav Magar, Barshi (IN); Satyanarayana Tallam, Kharghar, Raigad (IN); Prakash Ramnani, Kharghar, Navi Mumbai (IN); Srinivas Singh, Mumbai (IN); ArunKumar Pandey, Kharghar, Navi Mumbai (IN); Umadoss Pothuvan, Kharghar, Navi Mumbai (IN)

(21) Appl. No.: 18/655,810

(22) Filed: May 6, 2024

(30) Foreign Application Priority Data
May 8, 2023 (IN) 202321032496

(51) Int. Cl. A61K 9/48; A61K 31/4985

(52) U.S. Cl. CPC A61K 9/4866; A61K 9/485; A61K 9/4858; A61K 31/4985

(57) ABSTRACT

The present invention relates to a pharmaceutical composition for oral administration comprising: a) about 5% w/w to about 40% w/w of lumateperone or pharmaceutically acceptable salt thereof; b) about 50% w/w to about 95% w/w of at least one diluent; c) about 1% w/w to about 10% w/w of at least one disintegrant; d) about 0.1% w/w to about 2% w/w of at least one glidant; and e) about 0.5% w/w to about 5% w/w of a lubricant selected from the group consisting of sodium stearyl fumarate, stearic acid, and combinations thereof, wherein the amount of nitrosamine impurity after exposure of the pharmaceutical composition to 40° C./75% RH for a period of six months is less than the FDA acceptable intake limit of the nitrosamine impurity based on maximum daily dose of lumateperone.

### CROSS-REFERENCE TO RELATED APPLICATIONS

This application claims priority to and the benefit of Indian patent application No. 202321032496, filed May 8, 2023.

### FIELD OF THE INVENTION

The present invention relates to pharmaceutical compositions for oral administration containing lumateperone or a pharmaceutically acceptable salt thereof. The compositions of the invention provide improved stability especially during the manufacturing process to ensure reproducibility and quantitative accuracy. Moreover, nitrosamine impurities are eliminated or reduced in such compositions.

### BACKGROUND OF THE INVENTION

Lumateperone is an atypical antipsychotic, present as lumateperone tosylate salt with the chemical name 4-((6bR,10aS)-3-methyl-2,3,6b,9,10,10a-hexahydro-1H,7H-pyrido[3′,4′: 4,5] pyrrolo[1,2,3-de] quinoxalin-8-yl)-1-(4-fluoro-phenyl)-butan-1-one 4-methylbenzenesulfonate. Its molecular formula is C₃₁H₃₆FN₃O₄S, and its molecular weight is 565.71 g/mol with the following chemical structure shown below:

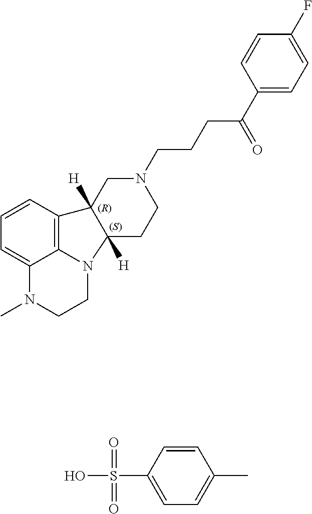

Currently lumateperone formulation marketed under tradename CAPLYTA® and used for Schizophrenia in adults and Depressive episodes associated with bipolar I or II disorder (bipolar depression) in adults, as monotherapy and as adjunctive therapy with lithium or valproate.

Lumateperone's mechanism of action involves simultaneous modulation of dopaminergic, serotonergic, and glutamatergic neurotransmission. Its dopaminergic actions involve pre-synaptic partial agonist and post-synaptic antagonist activity at dopamine D2 receptors. This dual action at the dopamine D2 receptor is unique to lumateperone compared to other antipsychotics.

U.S. Pat. No. RE39,680 discloses lumateperone and its pharmaceutically acceptable salts.

U.S. Pat. No. 10,464,938 discloses pharmaceutical composition comprising a toluenesulfonic acid addition salt of lumateperone i.e. lumateperone tosylate.

RE48,839 discloses method for the treatment of one or more 5-HT2A-related disorders such as for the treatment of one or more sleep disorders, depression, psychosis, dyskinesias, and/or Parkinson's disease or any combinations by lumateperone in free or pharmaceutically acceptable salt form.

U.S. Pat. No. 11,292,793 discloses solid state forms of Lumateperone p-Tosylate and processes for preparation thereof. It also discloses amorphous Lumateperone p-tosylate and a solid dispersion comprising amorphous Lumateperone p-tosylate and one or more pharmaceutically acceptable polymers and processes for preparation thereof.

U.S. Pat. Nos. 10,695,345 (“the '345 patent”); 11,052,084 (“the '084 patent”); 11,690,842 (“the '842 patent”); and 11,806,348 (“the '348 patent”) are listed in FDA's Approved Drug Products and Therapeutic Equivalence (“Orange Book”) for CAPLYTA® (lumateperone tosylate). The '345, '084, '842, and '348 patents discloses pharmaceutical capsule compositions comprising lumateperone, in free, or pharmaceutically acceptable salt form, and at least one diluent or carrier, including a filler, disintegrant, glidant, and/or lubricant. According to the '345, '084, '842, and '348 patents, in order to provide more stable lumateperone capsule compositions and prevent sticking of the pharmaceutical capsule compositions to the tamping pins in the capsule filling machine, the compositions must contain 60-90 weight percent of mannitol and 0.5-10 weight percent of croscarmellose sodium, as excipients, and be prepared using a dry powder blending process.

Additionally, nitrosamine impurities are possible in the synthesis of lumateperone and its pharmaceutical salt thereof. The Nitrosamine impurities may originate from any or all of the active pharmaceutical ingredient, excipients, pharmaceutical packaging materials, and/or manufacturing process and equipment. Examples of Nitrosamine impurities include, but are not limited to, N-Nitroso Lumateperone, LMT-1 Nitroso impurity, N-Nitrosodimethyl amine (“NDMA”), N-Nitrosodiethyl amine (“NDEA”), N-Nitrosodiisopropylamine “NDIPA”), N-Nitrosoethylisopropylamine (“NIPEA”), N-nitroso-N-methyl-4-aminobutyric acid (“NMBA”), N-Nitrosodibutylamine (“NDBA”), and N-Methyl-N-phenylnitrosamine (“NMPA”). Nitrosamines are generally formed when a secondary or tertiary amine reacts with a nitrosating agent. While the amounts of the Nitrosamine impurities in drugs is generally low, some levels have been above the U.S. Food and Drug Administration's acceptable daily limit, potentially exposing many people to a slightly increased risk of cancer. Because of the aforementioned health-related effects, stringent limitations have been placed upon the permissible amounts of nitrosamine impurities in drug products. Because of the exceptionally low allowable limits for nitrosamine impurities in these products it become important to control the nitrosamine impurities during synthesis of active pharmaceutical ingredient or drug formulation.

There is a need for improved pharmaceutical compositions in the form of solid oral dosages containing lumateperone which provide improved stability especially during the manufacturing process to ensure reproducibility and quantitative accuracy. Additionally, there is a need to develop pharmaceutically acceptable lumateperone compositions wherein the amount of nitrosamine impurity is less than the FDA acceptable intake limit of the nitrosamine impurity based on maximum daily dose of lumateperone.

### SUMMARY OF THE INVENTION

The invention provides a pharmaceutical composition for oral administration comprising: a) about 5% w/w to about 40% w/w of lumateperone or pharmaceutically acceptable salt thereof; b) about 50% w/w to about 95% w/w of at least one diluent; c) about 1% w/w to about 10% w/w of at least one disintegrant; d) about 0.1% w/w to about 2% w/w of at least one glidant; and e) about 0.5% w/w to about 5% w/w of a lubricant selected from the group consisting of sodium stearyl fumarate, stearic acid, and combinations thereof, wherein the amount of nitrosamine impurity after exposure of the pharmaceutical composition to 40° C./75% RH for a period of six months is less than the FDA acceptable intake limit of the nitrosamine impurity based on maximum daily dose of lumateperone.

Another aspect of the invention provides a pharmaceutical composition for oral administration comprising: a) about 5% w/w to about 40% w/w of lumateperone or pharmaceutically acceptable salt thereof; b) about 50% w/w to about 95% w/w of mannitol; c) about 1% w/w to about 10% w/w of croscarmellose sodium; d) about 0.1% w/w to about 2% w/w of colloidal silicon dioxide; and e) about 0.5% w/w to about 5% w/w of a lubricant selected from the group consisting of sodium stearyl fumarate, stearic acid, and combinations thereof.

Another aspect of the invention provides a pharmaceutical composition for oral administration comprising: a) about 5% w/w to about 40% w/w of lumateperone or pharmaceutically acceptable salt thereof; b) about 50% w/w to about 95% w/w of mannitol; c) about 1% w/w to about 10% w/w of croscarmellose sodium; d) about 0.1% w/w to about 2% w/w of colloidal silicon dioxide; and e) about 0.5% w/w to about 5% w/w of sodium stearyl fumarate; wherein the mannitol, colloidal silicon dioxide, and sodium stearyl fumarate are present as intragranular excipients, and the croscarmellose sodium, colloidal silicon dioxide, and sodium stearyl fumarate are present as extragranular excipients, wherein the total amount of the intragranular and extragranular excipients are within said weight percents.

Another aspect of the invention provides a process for preparing a pharmaceutical composition comprising the steps of: (a) mixing lumateperone or a pharmaceutically acceptable salt thereof, and at least one diluent, at least one glidant, and at least one lubricant to form a mixture; (b) granulating the mixture from Step (a) to form granules; (c) mixing at least one disintegrant, at least one lubricant, and at least one glidant with the granules formed in Step (b) to form a lubricated blend; and (d) encapsulating or tableting the lubricated blend from Step (c).

In a preferred embodiment of the invention, the lumateperone composition is in a capsule dosage form and prevents sticking of the composition to tamping pins in capsule filling machines, which otherwise would negatively affect the reproducibility and quantitative accurate filling of capsules during the manufacturing process.

### DETAILED DESCRIPTION OF THE INVENTION

The present invention can be more readily understood by reading the following description of the invention and study of the included examples.

The use of the word “a” or “an” when used in conjunction with the term “comprising” in the claims and/or the specification may mean “one,” but it is also consistent with the meaning of “one or more,” “at least one,” and “one or more than one.”

The term “about” as used herein means a deviation within 10%, more preferably within 5%, and even more preferably, within 2% of the numbers reported.

The term “stable” refers to formulations that substantially retain the labelled amount of the therapeutically active ingredient during storage for commercially relevant times, and the drug-related impurity contents in the formulations remain within acceptable limits.

The term “impurity” refers to undesired contents present or produced in a pharmaceutical composition.

The term “therapeutically effective amount” is defined to mean the amount or quantity of lumaterperone that is sufficient to elicit an appreciable pharmacological response, when administered to the patient.

The term “bioavailability” denotes the degree to which the lumaterperone becomes available to the target tissue after administration.

The term “excipient(s)” are components that are added to the pharmaceutical composition of the invention other than the lumateperone. Excipient(s) includes, but are not limited to, diluent, binder, disintegrant, lubricant/glidants, antioxidant, plasticizer, surfactants, solvent/vehicle, flavors or colorants, coating materials and any other excipient known to the art for making solid oral pharmaceutical compositions. The excipients that are useful in preparing the pharmaceutical composition of the invention are generally safe, inert, non-toxic and are acceptable for human use.

The term “core” as used herein, refers to an interior compartment of a unit dosage form.

The term “w/w” refers to total weight of active substance/excipients with respect to total composition weight or the proportion of a particular substance within a mixture, as measured by weight or mass.

The term “pharmaceutical composition” or “solid oral pharmaceutical composition” or “composition” or “formulation” refers to a solid dosage form comprising lumateperone suitable for administration such as a hard or soft gelatin capsule, tablet, caplet, mini-tablets, pellets, granules, pills, suspension and the like.

The term “granulation” as used herein, and as conventionally used in the pharmaceutical industry, refers to the act or process in which primary powder particles are made to adhere to form larger, multiparticle entities called granules. Granules may for example be formed collecting particles together by creating mechanical bonds between them, e.g. by compression or by using a binder. Granulation is extensively used in the manufacturing of tablets and capsules.

Preferably, the pharmaceutical compositions of the invention are prepared by dry granulation, wet granulation, direct compression, or dry powder blending. In one embodiment, the compression of a blend of granules including optional extragranular excipients, or a powder blend into a dosage form can be carried out using a conventional tableting machine, rotary compression machine, or automatic capsule filling machine.

Granulation is a process used to produce larger particles, known as granules, from material aggregations consisting of smaller primary particles, but without changing the phase condition of most of the solid particles. Granules are used in both the production of tablets and in capsule filling. The most widely used general method of granulation is wet granulation, though other granulation processes such as dry granulation, roller compaction, and melt granulation may be used. Granulation can be performed in a variety of equipment such as, but not limited to, low shear, high shear granulators, fluid bed granulator, roller compactor, and slugger.

The term “blending” refers to the mixing of pharmaceutical ingredients to form a mixture of the ingredients, e.g. active pharmaceutical ingredient (API) and diluent, as defined by pharmaceutical specifications in the compendial references using a variety of equipment such as, but not limited to, “V”-blenders, bin-blenders, cone-blenders.

The term “encapsulation” refers to a range of techniques used to enclose medicines in a shell, e.g. a two-piece capsule, such as a two-piece hard shell capsule. The capsule referred to herein may be taken orally. Capsules may be designed with a telescoping cap and body manufactured from e.g. gelatin or cellulose.

The capsules of the present invention include, for example, hard or soft gelatin capsule. Hard gelatin capsules are two-piece gel encapsulations of solid material. The capsule shell consists of two halves, an outer half and an inner half, which when joined and sealed form a secure enclosure for the material contained therein. The active pharmaceutical ingredient, i.e., lumateperone, may be comprised as a powder, or as one or more granules or pellets within the capsule. Soft gelatin capsules are single-piece gel encapsulations of solid material, and such solid material may be in the form of an aqueous gel.

The term “active ingredient” or “active agent” or “drug” used interchangeably, is defined to mean active drug i.e. lumateperone and its salts, solvate, esters, isomers, polymorphs that induce a desired pharmacological or physiological effect.

The term “lumateperone” includes all pharmaceutically acceptable salts, esters, isomers, stereo isomers, crystalline and amorphous forms. Pharmaceutically acceptable salts of lumateperone includes but not limited to tosylate, oxalate, phosphate or acid phosphate, hydrochloride, hydrobromide, sulphate, mesylate, acetate, maleate, fumarate, lactate, tartrate, citrate, methanesulfonate, pamoate, palmitate, and gluconate salts; most preferably used salt of lumateperone is tosylate salt.

Lumateperone or a pharmaceutically acceptable salt thereof exists mainly in crystalline and amorphous polymorphic forms. Crystalline forms have different arrangements and/or conformations of the molecules in the crystal lattice; and amorphous forms consist of disordered arrangements of molecules that do not possess a distinguishable crystal lattice; most preferably used polymorph of lumateperone or a pharmaceutically acceptable salt thereof is crystalline polymorph.

Lumateperone or pharmaceutically acceptable salt thereof may be present in an amount of about 5 to about 40% w/w, preferably about 15 to about 25% w/w, more preferably about 18 to 22% w/w of the composition.

The present invention provides a pharmaceutical composition comprising lumateperone base in an amount of about 1 to about 100 mg, preferably about 5 to about 70 mg, more preferably about 10.5 mg, 21 mg or 42 mg. Equivalent amount of pharmaceutically acceptable salt can be calculated accordingly. For instance, Lumateperone base in an amount of 42 mg is equivalent to about 60 mg of lumateperone tosylate.

Bulk, tapped density, and particle size distribution are factors to consider when evaluating a granulation or a powder blend for filling into capsules or compression into tablets. Preferably, the lumateperone or pharmaceutically acceptable salt thereof has a particle size in the range of 10 μm to 150 μm. Preferably, the D90 particle size is between 70 μm and 100 μm.

The present invention provides a pharmaceutical composition comprising lumateperone or pharmaceutically acceptable salt thereof and one or more pharmaceutically acceptable inactive ingredients, or excipients, that impart some function within the composition. Preferably, the selected excipient will be chemically and physically compatible with lumateperone and with each other, and known to be functionally useful in oral solid dosage forms. Generally, excipients are classified according to some primary function that they perform in the composition, and are broken down into categories such as diluents, binders, lubricants, antiadherents, glidants, disintegrants, release modifiers, surfactants, and miscellaneous components such as buffers, adsorbents, and stabilizers. Occasionally, an excipient will serve more than one function within a tablet or capsule, and the function a particular excipient serves may differ between tablets and capsules.

Another aspect of the invention, the pharmaceutical composition comprises lumateperone or a pharmaceutically acceptable salt thereof and pharmaceutically acceptable excipients selected from the group consisting of diluents, disintegrants, lubricants, glidants, antioxidants, flavouring agent, colouring agent and sweetening agent.

In one aspect of the invention, an oral stable pharmaceutical capsule composition comprising about 5 to about 40% w/w of lumateperone or pharmaceutically acceptable salt thereof and one or more pharmaceutically acceptable excipients.

In another aspect, there is provided a pharmaceutical composition comprising: a) about 5 to about 40% w/w of lumateperone or pharmaceutically acceptable salt thereof; b) about 60 to about 95% w/w of pharmaceutically acceptable excipients; wherein, the pharmaceutical composition is in the form of tablet or capsule.

Diluents according to the present invention are selected from, but not limited to, silicon dioxide, titanium dioxide, talc, powdered cellulose, microcrystalline cellulose, dicalcium phosphate, mannitol, sorbitol or other sugar alcohols, isomalt, sucrose, lactose, and the like used either alone or in combinations thereof and the most preferably used diluent is mannitol. The present inventors have determined that mannitol has poor flow and binding properties, making it poor as a direct compression excipient. Accordingly, higher lubricant quantities were needed for mannitol-containing granulations compared to those made with other diluents. Diluent may be present in an amount of about 50% w/w to about 95% w/w, preferably about 65% w/w to about 85% w/w, more preferably about 72% w/w to about 76% w/w of the composition.

Disintegrants according to the present invention are selected from, but not limited to, croscarmellose sodium, crospovidone, carmellose calcium, sodium starch glycolate, polacrilin potassium, starches, substituted hydroxypropyl cellulose, powdered agar and like used either alone or in combinations thereof and the most preferably used disintegrant is croscarmellose sodium.

Disintegrant can be added intragranular or extragranular. The disintegration test basically consists of placing a dosage form in an immersion medium under defined experimental conditions and measuring the time taken for the dosage form to disintegrate. The time in which the tablet or capsule should disintegrate is defined in the applicable monograph. Disintegrant may be present in an amount of about 1% w/w to about 10% w/w, preferably about 1.5% w/w to about 7% w/w, more preferably about 2% w/w to about 3% w/w. Disintegrant can be added intragranular or extragranular however extragranular addition may result in an improved dissolution.

Lubricants according to the present invention are selected from, but not limited to, magnesium stearate, calcium stearate, aluminium stearate, sucrose stearate, sucrose fatty acid ester, stearic acid, fumaric acid, palmitic acid, sodium stearyl fumarate, polyethylene glycol, talc, and the like used either alone or in combinations thereof and the most preferably used lubricant is sodium stearyl fumarate or mixture of sodium stearyl fumarate and stearic acid. Lubricant can be added intragranular or extragranular and may be present in an amount of about 0.5% w/w to about 5% w/w, preferably about 1% w/w to about 4% w/w, more preferably about 1.5% w/w to about 3% w/w of the composition.

Glidants according to the present invention are selected from, but not limited to, colloidal silicon dioxide, calcium silicate, magnesium silicate, silicon hydrogel, cornstarch, talc, and the like used either alone or in combinations thereof and the most preferably used glidant is colloidal silicon dioxide. Glidants can be added intragranular or extragranular and may be present in an amount of about 0.1% w/w to about 2% w/w, preferably about 0.5% w/w to about 1.5% w/w, more preferably about 1% w/w of the composition.

For capsules that are filled with granulated lumateperone, binders may also be used in preparing the granules. Binders can be used in solution or in powder form. Binders serve to assist in the agglomeration processes in granulation, and can include gelatin, starch and starch derivatives, sucrose, dextrin, acacia gum, methylcellulose, carboxymethylcellulose sodium, ethylcellulose, hydroxypropyl cellulose (HPC), polyvinylpyrrolidone (PVP or Povidone), polyethylene glycol (PEG) and others. Unexpectedly, inventors of the present invention have found that intragranular and/or extragranular addition of sodium stearyl fumarate in combination with colloidal silicon dioxide along with intragranular single step addition of mannitol has resulted in an improved dissolution rate, stability, and manufacturing process.

Antioxidants are used to prevent oxidation, thereby preventing the deterioration of the preparation. Suitable antioxidants for use in the present invention include but not limited to propyl gallate, butylated hydroxyanisole (BHA), butylated hydroxytoluene (BHT), and combinations thereof.

“Plasticizer” refers to an excipient that can impart flexibility and/or stretchability to a coat or membrane. Examples of plasticizers include but are not limited to medium chain triglycerides, phthalates, phosphates, glycerol, citrates, adipates, sebacates, succinates, glycolates, and the like used either alone or in combinations thereof.

“Surfactant” refers to one or more excipients that may be added to the pharmaceutical composition to facilitate dissolution of poorly soluble excipients and/or to increase dissolution rate of composition or components thereof. Examples of surfactants include but are not limited to sodium lauryl sulphate, glycerol monostearate, sorbitan monolaurate, tween 60, tween 80 (polysorbate 80) etc.

A “Solvent/vehicle” is a substance that dissolves a solute (a chemically distinct liquid, solid or gas), resulting in a solution. Examples include but not limited to purified water, alcoholic solvents methanol, ethanol; ketones such as acetone, propanone; esters such as ethyl acetate, n-propyl acetate, isopropylacetate and nbutyl acetate and the like; ethers such as dimethylether, diethylether, methyltertiarybutylether, ethylmethylether, diisopropylether, and dioxane.

“Flavorants” refers to a substance that gives another substance flavor, altering the characteristics of the solute, causing it to become sweet, sour, etc. Pharmaceutically acceptable flavors includes but not limited to cherry, orange, vanilla.

Sweetening agents may be used to mask unpleasant taste or to achieve a desired taste. Examples of sweetening agents are glucose, sucrose, fructose, sorbitol, glycerol, cyclamate, aspartame, acesulfame potassium and neohesperidin dihydrochalcon.

In some embodiments, coloring agents may be used to introduce a uniformity of appearance to the product and/or to protect any light-sensitive ingredients. Suitable coloring agents include all pigments, dyes and lakes approved by the U.S. Food and Drug Administration (e.g., FD&C colorants), including but not limited to FD&C Yellow #6, FD&C Blue #1, FD&C Red #3, black iron oxide, red iron oxide, iron oxide yellow, tartrazine, erythrosine, amaranth lake, titanium dioxide, opadry systems, or any combination thereof. Coloring agents may be included within the capsule shell or within the capsule fill, or both, also coloring agents added for the coating of the tablet.

One embodiment of the invention provides a pharmaceutical composition comprising about 5% w/w to about 40% w/w of lumateperone or pharmaceutically acceptable salt thereof, about 50% w/w to about 95% w/w of diluent, about 1% w/w to about 10% w/w of disintegrant, about 0.1% w/w to about 2% w/w of glidant and about 0.5% w/w to about 5% w/w of lubricant.

One embodiment of the invention provides a pharmaceutical composition comprising about 5% w/w to about 40% w/w of lumateperone or pharmaceutically acceptable salt thereof, about 50% w/w to about 95% w/w of diluent, about 1% w/w to about 10% w/w of disintegrant, about 0.1% w/w to about 2% w/w of glidant, and about 0.5% w/w to about 5% w/w of lubricant; wherein the pharmaceutical composition is stable for at least 24 hours.

One embodiment of the invention provides a pharmaceutical composition comprising: a) about 5% w/w to about 40% w/w of lumateperone or pharmaceutically acceptable salt thereof; b) about 50% w/w to about 95% w/w of mannitol; c) about 1% w/w to about 10% w/w of croscarmellose sodium; d) about 0.1% w/w to about 2% w/w of colloidal silicon dioxide; e) about 0.5% w/w to about 5% w/w of sodium stearyl fumarate; and f) optionally other pharmaceutically acceptable excipients; wherein, said pharmaceutical composition is in the form of capsule or tablet.

One embodiment of the invention provides a pharmaceutical composition comprising: a) about 5% w/w to about 40% w/w of lumateperone or pharmaceutically acceptable salt thereof; b) about 50% w/w to about 95% w/w of mannitol; c) about 1% w/w to about 10% w/w of croscarmellose sodium; d) about 0.1% w/w to about 2% w/w of colloidal silicon dioxide; e) about 0.5% w/w to about 5% w/w of sodium stearyl fumarate; and f) optionally other pharmaceutically acceptable excipients.

One embodiment of the invention provides a pharmaceutical composition comprising: a) about 20% w/w of lumateperone tosylate; b) about 74% w/w of mannitol; c) about 2.7% w/w of croscarmellose sodium; d) about 1% w/w of colloidal silicon dioxide; e) about 2% w/w of sodium stearyl fumarate; and f) optionally other pharmaceutically acceptable excipients.

One embodiment of the invention provides a pharmaceutical composition comprising about 5 to about 70 mg of lumateperone or pharmaceutically acceptable salt thereof and about 0.1 to about 5 mg of colloidal silicon dioxide.

One embodiment of the invention provides a pharmaceutical composition comprising about 5 to about 70 mg of lumateperone or pharmaceutically acceptable salt thereof and about 0.1 to about 5 mg of colloidal silicon dioxide wherein about 50% of colloidal silicon dioxide is present in intragranular phase and about 50% of colloidal silicon dioxide is present in extragranular phase.

One embodiment of the invention provides a pharmaceutical composition comprising about 5 to about 70 mg of lumateperone or pharmaceutically acceptable salt thereof and about 0.5 to about 8 mg of sodium stearyl fumarate.

One embodiment of the invention provides a pharmaceutical composition comprising about 5 to about 70 mg of lumateperone or pharmaceutically acceptable salt thereof and about 0.5 to about 8 mg of sodium stearyl fumarate wherein about 50% of sodium stearyl fumarate is present in intragranular phase and about 50% of sodium stearyl fumarate is present in extragranular phase.

One embodiment of the invention provides a pharmaceutical composition comprising: a) about 5 to about 70 mg of lumateperone or pharmaceutically acceptable salt thereof; b) about 25 to about 250 mg of mannitol; c) about 1 to about 25 mg of croscarmellose sodium; d) about 0.1 to about 5 mg of colloidal silicon dioxide; e) about 0.5 to about 8 mg of sodium stearyl fumarate; and f) optionally other pharmaceutically acceptable excipients; wherein, said pharmaceutical composition is in the form of capsule or tablet.

One embodiment of the invention provides a pharmaceutical composition comprising: a) about 5 to about 70 mg of lumateperone or pharmaceutically acceptable salt thereof; b) about 25 to about 250 mg of mannitol; c) about 1 to about 25 mg of croscarmellose sodium; d) about 0.1 to about 5 mg of colloidal silicon dioxide; e) about 0.5 to about 8 mg of lubricants selected from the group consisting of sodium stearyl fumarate, stearic acid or mixture thereof; and f) optionally other pharmaceutically acceptable excipients.

One embodiment of the invention provides a pharmaceutical composition in the form of capsule dosage form comprising: a) about 60 mg of lumateperone tosylate equivalent to about 42 mg of lumateperone; b) about 223 mg of mannitol; c) about 8 mg of croscarmellose sodium; d) about 3 mg of colloidal silicon dioxide; e) about 6 mg of sodium stearyl fumarate; and f) optionally other pharmaceutically acceptable excipients.

One embodiment of the invention provides a pharmaceutical composition in the form of capsule dosage form comprising: a) about 60 mg of lumateperone tosylate equivalent to about 42 mg of lumateperone; b) about 223 mg of mannitol; c) about 8 mg of croscarmellose sodium; d) about 3 mg of colloidal silicon dioxide; e) about 6 mg of sodium stearyl fumarate; and f) optionally other pharmaceutically acceptable excipients wherein about 50% of sodium stearyl fumarate or colloidal silicon dioxide is present in intragranular phase and about 50% of sodium stearyl fumarate or colloidal silicon dioxide is present in extragranular phase.

One embodiment of the invention provides a pharmaceutical composition in the form of capsule dosage form comprising: a) about 30 mg of lumateperone tosylate equivalent to about 21 mg of lumateperone; b) about 111 mg of mannitol; c) about 4 mg of croscarmellose sodium; d) about 1.5 mg of colloidal silicon dioxide; e) about 3 mg of sodium stearyl fumarate; and f) optionally other pharmaceutically acceptable excipients.

One embodiment of the invention provides a pharmaceutical composition in the form of capsule dosage form comprising: a) about 30 mg of lumateperone tosylate equivalent to about 21 mg of lumateperone; b) about 111 mg of mannitol; b) about 4 mg of croscarmellose sodium; c) about 1.5 mg of colloidal silicon dioxide; d) about 3 mg of sodium stearyl fumarate; and f) optionally other pharmaceutically acceptable excipients; wherein about 50% of sodium stearyl fumarate or colloidal silicon dioxide is present in intragranular phase and about 50% of sodium stearyl fumarate or colloidal silicon dioxide is present in extragranular phase.

One embodiment of the invention provides a pharmaceutical composition in the form of capsule dosage form comprising: a) about 15 mg of lumateperone tosylate equivalent to about 10.5 mg of lumateperone; b) about 56 mg of mannitol; b) about 2 mg of croscarmellose sodium; c) about 0.75 mg of colloidal silicon dioxide; d) about 1.5 mg of sodium stearyl fumarate; and f) optionally other pharmaceutically acceptable excipients.

One embodiment of the invention provides a pharmaceutical composition in the form of capsule dosage form comprising: a) about 15 mg of lumateperone tosylate equivalent to about 10.5 mg of lumateperone; b) about 56 mg of mannitol; b) about 2 mg of croscarmellose sodium; c) about 0.75 mg of colloidal silicon dioxide; d) about 1.5 mg of sodium stearyl fumarate; and f) optionally other pharmaceutically acceptable excipients; wherein about 50% of sodium stearyl fumarate or colloidal silicon dioxide is present in intragranular phase and about 50% of sodium stearyl fumarate or colloidal silicon dioxide is present in extragranular phase.

In reference to a process for preparing the pharmaceutical compositions of the invention, the terms screen, screening, delump, delumping, milling, and sizing are used interchangeably herein. These terms refer to separation according to size.

One embodiment of the invention provides a process for preparing the pharmaceutical compositions of the invention, wherein the process comprises the steps of: (a) mixing lumateperone or a pharmaceutically acceptable salt thereof, and at least one diluent, at least one glidant, and at least one lubricant to form a mixture; (b) granulating the mixture from Step (a) to form granules; (c) mixing at least one disintegrant, at least one lubricant, and at least one glidant with the granules formed in Step (b) to form a lubricated blend; and (d) encapsulating or tableting the lubricated blend from Step (c).

One embodiment of the invention provides a process for preparing the pharmaceutical composition of the invention, wherein the process comprises the steps of: (i) mixing lumateperone or a pharmaceutically acceptable salt thereof, and at least one diluent, to form a mixture; (ii) adding at least one glidant and at least one lubricant to the mixture from Step (i), to form a blend of particles; (iii) mixing at least one disintegrant, at least one lubricant, and at least one glidant with the blend of particles from Step (ii), to form a lubricated blend of particles; and (iv) encapsulating or tableting the lubricated blend of particles from Step (iii). Optionally, the process includes a sifting step of sifting the lubricant prior to mixing the sifted lubricant with the blend of particles from Step (ii).

One embodiment of the invention provides a process for preparing the pharmaceutical compositions of the invention, wherein the process comprises the steps of: (I) mixing lumateperone or a pharmaceutically acceptable salt thereof, and at least one diluent, to form a mixture; (II) adding at least one glidant to the mixture from Step (I), to form a combination of particles; (III) sifting the combination of particles from Step (II) to form a sifted mixture of particles; (IV) milling the sifted mixture of particles from Step (III), to form milled particles; (V) mixing the milled particles from Step (IV) with at least one disintegrant, at least one lubricant, and at least one glidant, to form a lubricated blend of particles; and (VI) encapsulating or tableting the lubricated blend of particles from Step (V). Optionally, the process includes a further sifting step of sifting the lubricant prior to mixing with the milled particles from Step (IV).

One embodiment of the invention provides a process for preparing the pharmaceutical compositions of the invention, wherein the process comprises the steps of: (A) preparing a mixture of lumateperone or a pharmaceutically acceptable salt thereof, at least one diluent, at least one glidant, and at least one lubricant; (B) granulating the mixture of Step (A) to form intragranular particles; (C) mixing at least one disintegrant, at least one lubricant, and at least one glidant with the intragranular particles from Step (B) to form a mixture of intragranular particles and extragranular particles; and (D) encapsulating or tableting the mixture of intragranular and extragranular particles from Step (C). Optionally, the process includes a further sifting step of sifting the lubricant prior to mixing the lubricant with the intragranular particles from Step (B).

One embodiment of the invention provides a process for preparing a capsule composition, wherein the process comprises steps of: a) Dispensing of required raw material in appropriate quantities; b) Mixing of lumateperone or its pharmaceutically acceptable salts and mannitol followed by sifting of mixed materials, croscarmellose sodium and colloidal silicon dioxide through #30 screen; c) Pre-blending: Mixing the above sifted materials in a blender for 20 min at 22 RPM; d) Milling: Passing the blended material through Cone mill fitted with 0.5 mm screen; e) Blending: Mixing the above milled blend in a blender for 10 min at 22 RPM; f) Lubrication: Sifting sodium stearyl fumarate through #60, loading of sifted sodium stearyl fumarate in blender; g) Capsule Filling: Filling the lubricated blend in hard gelatin capsules; wherein; mannitol is added in a single step for preparation of granules.

One embodiment of the invention provides a process for preparing a capsule composition, wherein the process comprises steps of: a) Dispensing of required raw material in appropriate quantities and pass the materials through cone mill with 1 mm screen; b) Pre-blending: Mixing of lumateperone or its pharmaceutically acceptable salts, mannitol and colloidal silicon dioxide in a blender for 10 min at suitable speed; c) Milling: Passing the blended material through Cone mill fitted with 1 mm screen; d) Blending: Mixing the above milled blend in a blender for 10 min at suitable speed; e) Lubrication: Loading of sifted Sodium Stearyl fumarate and/or stearic acid in blender to obtain lubricated blend; f) Roller compaction and sizing: Dry granulation of lubricated blend using roller compacter and sizer to obtain dried granules; g) Blending: Mixing of croscarmellose sodium and Colloidal silicon dioxide with the sized granules in a blender for 10 minutes at suitable speed; h) Lubrication: Loading of sifted extra-granular sodium stearyl fumarate and/or stearic acid in the blend to obtain final lubricated blend; i) Capsule Filling: Filling the final lubricated blend in hard gelatin capsules.

In one embodiment of the processes of the invention, a diluent, preferably mannitol, is added completely in a single intragranular step.

One embodiment of the invention provides a pharmaceutical composition comprising: an intragranular phase comprising lumateperone tosylate, mannitol, colloidal silicon dioxide and sodium stearyl fumarate, and an extragranular phase comprising croscarmellose sodium, colloidal silicon dioxide and a sodium stearyl fumarate; wherein at least part of the colloidal silicon dioxide and sodium stearyl fumarate are present in the granules (intragranular) and at least part of the colloidal silicon dioxide and sodium stearyl fumarate are extragranular.

One embodiment of the invention provides a pharmaceutical composition comprising: a) about 5 to about 70 mg of lumateperone or pharmaceutically acceptable salt thereof; b) about 25 to about 250 mg of mannitol; c) about 1 to about 25 mg of croscarmellose sodium; d) about 0.1 to about 5 mg of colloidal silicon dioxide; e) about 0.5 to about 8 mg of sodium stearyl fumarate; and f) optionally other pharmaceutically acceptable excipients; wherein at least part of the colloidal silicon dioxide and sodium stearyl fumarate are present in the granules (intragranular) and at least part of the colloidal silicon dioxide and sodium stearyl fumarate are extragranular.

One embodiment of the invention provides compositions comprising crystalline lumateperone particles having a D90 of 10 μm or more. In a preferred embodiment, the D90 particle size of lumateperone is in the range of 10 to 150 μm. Preferably, the D90 particle size of lumateperone is between 70 and 100 μm. The particle size distribution, and/or bulk density of the granulated lumateperone may be controlled and matched to other excipient(s) to improve flow of the composition and assure content uniformity and low variability of the product. Additionally matching of excipient(s) allows reproducibility during encapsulation of lumateperone. The matching of physical properties of the lumateperone and other excipients used herein enables pharmaceutical manufacturing, in particular capsule filling at sufficient speed such as more than 40,000 capsules per hour. Matching of the lumateperone may for example be done by matching the particle size distribution of the lumateperone and one or more excipient. The bulk density of the lumateperone and the one or more excipients may also be matched.

The pharmaceutical compositions of the invention are subjected to tests such as dissolution, assay, impurity profiling, related substances, content uniformity, blend uniformity, hardness, thickness, friability, bulk density, tapped density, Hausner ratio (HR), compressibility index, particle size distribution, and loss on drying (LOD).

The pharmaceutical compositions of the invention may be used in any solid dosage form including immediate release (IR) or extended release (ER) of various strengths. Suitable dosage forms include, but are not limited to, tablets, coated tablets, layered tablets, granules, powders, microparticles, capsules which may be hard gelatin or soft gelatin, caplets, sachets, pellets, spheroids, mini-tablets, beads, microcapsules and pills. The compositions of the invention can be packed into suitable packaging system e.g., blister packs, strip packs, alu-alu packs, bottles such as glass and plastic bottles, etc. Preferably, the bottles used are composed of plastic, typically high-density polyethylene (HDPE). Many of products packaged in HDPE bottles are moisture sensitive. A drying agent (referred to as a desiccant) e.g. silica gel, may be incorporated into the bottle during packaging.

One embodiment of the invention provides a pharmaceutical composition comprising lumateperone or pharmaceutically acceptable salt used for the treatment of one or more sleep disorders, depression, psychosis, dyskinesias, and/or Parkinson's disease or any combinations.

One embodiment of the invention provides a pharmaceutical composition comprising lumateperone or pharmaceutically acceptable salt used for the treatment of schizophrenia in adults and Depressive episodes associated with bipolar I or II disorder (bipolar depression) in adults, as monotherapy and as adjunctive therapy with lithium or valproate.

One embodiment of the invention provides a method of treating schizophrenia in adults and Depressive episodes associated with bipolar I or II disorder (bipolar depression) in adults, as monotherapy and as adjunctive therapy with lithium or valproate by a pharmaceutical composition comprising lumateperone or pharmaceutically acceptable salt.

One embodiment of the invention provides a use of pharmaceutical composition comprising lumateperone or pharmaceutically acceptable salt for the treatment of schizophrenia in adults and Depressive episodes associated with bipolar I or II disorder (bipolar depression) in adults, as monotherapy and as adjunctive therapy with lithium or valproate.

The invention also provides a novel process for the elimination or reduction of nitrosamine impurities in the compositions containing lumateperone or pharmaceutical salts thereof, wherein the compositions are exposed to one or more desiccants in closed containers. Suitable desiccants are selected from a group consisting of silica desiccants, oxygen absorbing desiccants, activated carbon desiccants. The desiccants are further selected from a group comprising of Stabilox H-60, Stabilox H-42, molecular sieves, silica gels, activated carbon and mixtures thereof.

The lumateperone compositions of the invention were exposed to one or more desiccants while being stored in closed 30's HDPE Bottle, 100's HDPE Bottle, or 10's Alu-Alu Blister for a period of six months at 25° C./60% RH or six months at 40° C./75% RH. The stability results for the lumateperone compositions of the invention show that there is no significant increase in the nitrosamine impurities when the drug product is stored even after six months of being exposed to accelerated storage conditions. Advantages of the process of the invention for eliminating or reducing nitrosamine impurities in the lumateperone compositions include: (i) simple and quick; (ii) easily scaled up; (iii) economical; and (iv) avoids multiple techniques for nitrosamine impurity reduction.

Another aspect of the invention provides a pharmaceutical composition comprising lumateperone or pharmaceutically acceptable salt thereof, wherein the lumateperone is prepared by a process which avoids the formation of nitrosoamine impurities by avoiding using certain amines and nitrites in the synthesis of lumateperone, which are prone to form nitrosamine impurities.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### EXAMPLES

### Example 1—Capsule Composition (42 mg) Prepared by Dry Powder Blending

| | | Ex. 1A | Ex. 1B | Ex. 1C |
|---|---|---|---|---|
| Sr. No. | Ingredient | (mg/capsule) | | |
| A. | Dry mixing | | | |
| 1. | Lumateperone Tosylate | 60.38 | 60.38 | 60.38 |
| 2. | Mannitol | 215.82 | 217.32 | 217.82 |
| 3. | Croscarmellose Sodium | 17.5 | 16.00 | 15 |
| 4. | Colloidal silicon dioxide | 1.8 | 1.80 | 2 |
| B. | Lubrication | | | |
| 5. | Sodium Stearyl Fumarate | 4.7 | 4.50 | 4.8 |
| | Total weight | 300 | 300 | 300 |

The manufacturing process used for Example 1 was as follows:
1. Dispensing of required raw material in appropriate quantities;
2. Mixing of drug and mannitol followed by sifting of mixed materials through #30;
3. Sifting of croscarmellose sodium and colloidal silicon dioxide through #30;
4. Pre-blending: Mixing the above sifted materials in a blender for 20 min at 22 rpm;
5. Milling: Passing the blended material through Cone mill fitted with 0.5 mm screen;
6. Blending: Mixing the above milled blend in a blender for 10 min at 22 rpm;
7. Lubrication: Sifting sodium stearyl fumarate through #60, and loading of sifted sodium stearyl fumarate in a blender; and
8. Capsule Filling: Filling the lubricated blend in hard gelatin capsules.

### Example 2—Capsule Composition (21 mg) Prepared by Dry Powder Blending

| | | Ex. 2A | Ex. 2B | Ex. 2C |
|---|---|---|---|---|
| Sr. No. | Ingredient | (mg/capsule) | | |
| A. | Dry mixing | | | |
| 1. | Lumateperone Tosylate | 30.19 | 30.19 | 30.19 |
| 2. | Mannitol | 107.56 | 108.66 | 107.66 |
| 3. | Croscarmellose Sodium | 8.8 | 8.00 | 9 |
| 4. | Colloidal silicon dioxide | 1 | 0.90 | 0.9 |
| B. | Lubrication | | | |
| 5. | Sodium Stearyl Fumarate | 2.35 | 2.25 | 2.25 |
| | Total weight | 150 | 150 | 150 |

### The

capsule compositions in Example 2 were prepared using the manufacturing process as described in Example 1.

### Example 3—Capsule Composition (10.5 mg) Prepared by Dry Powder Blending

| | | Ex. 3A | Ex. 3B | Ex. 3C |
|---|---|---|---|---|
| Sr. No. | Ingredient | (mg/capsule) | | |
| A. | Dry mixing | | | |
| 1. | Lumateperone Tosylate | 15.1 | 15.10 | 15.1 |
| 2. | Mannitol | 53.53 | 54.33 | 53.33 |
| 3. | Croscarmellose Sodium | 4.5 | 4.00 | 4.64 |
| 4. | Colloidal silicon dioxide | 0.55 | 0.45 | 0.6 |
| B. | Lubrication | | | |
| 5. | Sodium Stearyl Fumarate | 1.32 | 1.125 | 1.33 |
| | Total weight | 75 | 75 | 75 |

The capsule compositions in Example 3 were prepared using the manufacturing process as described in Example 1.

### Example 4—Capsule Composition (42 mg) Prepared by Dry Granulation

| | | Ex. 4A | Ex. 4B | Ex. 4C |
|---|---|---|---|---|
| Sr. No. | Ingredient | (mg/capsule) | | |
| A | Dry Granulation | | | |
| 1 | Lumateperone Tosylate | 60.379 | 60.379 | 60.379 |
| 2 | Mannitol | 221.221 | 222.621 | 221.721 |
| 3 | Colloidal silicon dioxide | 1.8 | 1.5 | 1.8 |
| 4 | Sodium stearyl fumarate | 2.9 | 3 | 2.9 |
| B | Blending | | | |
| 5 | Croscarmellose sodium | 9 | 8 | 8.5 |
| 6 | Colloidal silicon dioxide | 1.8 | 1.5 | 1.8 |
| C | Lubrication | | | |
| 7 | Sodium stearyl fumarate | 2.9 | 3 | 2.9 |

| | | | |
|---|---|---|---|
| Total weight | 300 | 300 | 300 |

The manufacturing process used for Example 4 was as follows:
1. Dispensing of required raw material in appropriate quantities and pass the materials through cone mill with 1 mm screen;
2. Pre-blending: Mixing of lumateperone or its pharmaceutically acceptable salts, mannitol and colloidal silicon in a blender for 10 min at at 22 rpm;
3. Milling: Passing the blended material through Cone mill fitted with 1 mm screen;
4. Blending: Mixing the above milled blend in a blender for 10 min at suitable speed;
5. Lubrication: Loading of sifted sodium stearyl fumarate in a blender to obtain a lubricated blend;
6. Roller compaction and sizing: Dry granulation of lubricated blend using roller compacter and sizer to obtain dried granules;
7. Blending: Mixing of croscarmellose sodium and colloidal silicon dioxide with the sized granules in a blender for 10 minutes at 22 rpm;
8. Lubrication: Loading of sifted extragranular sodium stearyl fumarate in the blend to obtain a final lubricated blend; and
9. Capsule Filling: Filling the final lubricated blend into hard gelatin capsules.

### Example 5—Capsule Composition (21 mg) Prepared by Dry Granulation

| | | Ex. 5A | Ex. 5B | Ex. 5C |
|---|---|---|---|---|
| Sr. No. | Ingredient | (mg/capsule) | | |
| A | Dry Granulation | | | |
| 1 | Lumateperone Tosylate | 30.19 | 30.19 | 30.19 |
| 2 | Mannitol | 111.91 | 111.31 | 110.910 |
| 3 | Colloidal silicon dioxide | 0.85 | 0.75 | 0.75 |
| 4 | Sodium stearyl fumarate | 1.25 | 1.5 | 1.6 |
| B | Blending | | | |
| 5 | Croscarmellose sodium | 3.7 | 4 | 4.2 |
| 6 | Colloidal silicon dioxide | 0.85 | 0.75 | 0.75 |
| C | Lubrication | | | |
| 7 | Sodium stearyl fumarate | 1.25 | 1.5 | 1.6 |

| | | | |
|---|---|---|---|
| Total weight | 150 | 150 | 150 |

The capsule compositions in Example 5 were prepared using the manufacturing process as described in Example 4.

### Example 6—Capsule Composition (10.5 mg) Prepared by Dry Granulation

| | | Ex. 6A | Ex. 6B | Ex. 6C |
|---|---|---|---|---|
| Sr. No. | Ingredient | (mg/capsule) | | |
| A | Dry Granulation | | | |
| 1 | Lumateperone Tosylate | 15.095 | 15.095 | 15.095 |
| 2 | Mannitol | 55.675 | 55.655 | 55.955 |
| 3 | Colloidal silicon dioxide | 0.365 | 0.375 | 0.355 |
| 4 | Sodium stearyl fumarate | 0.65 | 0.75 | 0.75 |
| B | Blending | | | |
| 5 | Croscarmellose sodium | 2.2 | 2 | 1.74 |
| 6 | Colloidal silicon dioxide | 0.365 | 0.375 | 0.355 |
| C | Lubrication | | | |
| 7 | Sodium stearyl fumarate | 0.65 | 0.75 | 0.75 |

| | | | |
|---|---|---|---|
| Total weight | 75 | 75 | 75 |

The capsule compositions in Example 6 were prepared using the manufacturing process as described in Example 4.

### Example 7—Capsule Composition (42 mg) Prepared by Dry Granulation

| | | Ex. 7A | Ex. 7B | Ex. 7C |
|---|---|---|---|---|
| Sr. No. | Ingredient | (mg/capsule) | | |
| A | Dry Granulation | | | |
| 1 | Lumateperone Tosylate | 60.379 | 30.19 | 15.095 |
| 2 | Mannitol | 221.621 | 111.31 | 55.655 |
| 3 | Colloidal silicon dioxide | 1.5 | 0.75 | 0.375 |
| 4 | Sodium stearyl fumarate | 1.5 | 0.75 | 0.375 |
| 5 | Stearic acid | 1.5 | 0.75 | 0.375 |
| B | Blending | | | |
| 6 | Croscarmellose sodium | 8 | 4 | 2 |
| 7 | Colloidal silicon dioxide | 1.5 | 0.75 | 0.375 |
| C | Lubrication | | | |
| 8 | Sodium stearyl fumarate | 1.5 | 0.75 | 0.375 |
| 9 | Stearic acid | 1.5 | 0.75 | 0.375 |

| | | | |
|---|---|---|---|
| Total weight | 300 | 150 | 75 |

The manufacturing process used for Example 7 was as follows:
1. Dispensing of required raw material in appropriate quantities and pass the materials through cone mill with 1 mm screen;
2. Pre-blending: Mixing of lumateperone or its pharmaceutically acceptable salts, mannitol and colloidal silicon in a blender for 10 min at 22 rpm;
3. Milling: Passing the blended material through Cone mill fitted with 1 mm screen;
4. Blending: Mixing the above milled blend in a blender for 10 min at suitable speed;
5. Lubrication: Loading of sifted sodium stearyl fumarate and stearic acid in a blender to obtain a lubricated blend;
6. Roller compaction and sizing: Dry granulation of lubricated blend using roller compacter and sizer to obtain dried granules;
7. Blending: Mixing of croscarmellose sodium and colloidal silicon dioxide with the sized granules in a blender for 10 minutes at 22 rpm;
8. Lubrication: Loading of sifted extragranular sodium stearyl fumarate and stearic acid in the blend to obtain a final lubricated blend; and
9. Capsule Filling: Filling the final lubricated blend into hard gelatin capsules.

### Example 8—Dissolution and Stability Testing

Dissolution testing: The dissolution test is performed in accordance with general rules of USP for dissolution testing. The dissolution test parameters were: dissolution apparatus-USP II (paddle apparatus) with helix sinker; dissolution media—0.1 N HCl, dissolution volume-500 mL; paddle rotation speed—50 RPM.

Stability Study: Stability study conducted on compositions in order to ensure the stable composition for at least a period of six months.

TABLE 1
Dissolution testing and Stability study results of the
lumateperone compositions prepared in Example 4B.

| | | Storage condition (25° C./60% RH) | | |
|---|---|---|---|---|
| Sr. No. | Attributes | Initial | 3 Months | 6 Months |
| 1 | Assay (%) | 97.9 | 100.1 | 99.4 |
| 2 | Water content (%) | 0.2 | 0.2 | 0.2 |
| 3 | Dissolution (%) | 96 | 97 | 101 |
| | (0.1N HCl, 500 ml, | | | |
| | Paddle, 50 rpm, | | | |
| | with [symbol] | | | |
| 4 | Organic impurities (%) | | | |
| 4.1 | LMT1 impurities | Below LOQ | Below LOQ | Below LOQ |
| 4.2 | Cyclopropane | ND | ND | ND |
| | impurity | | | |
| 4.3 | Desfluoro impurity | Below LOQ | Below LOQ | Below LOQ |
| 4.4 | Dimer impurity | Below LOQ | Below LOQ | Below LOQ |
| 4.5 | Any unspecified | 0.02 | 0.03 | 0.07 |
| | degradation | | | |
| | product [symbol] | | | |
| 4.6 | Total degradation | 0.1 | 0.2 | 0.3 |

LOQ: Limit of Quantitation; ND: Not Detected; BDL: Below detection limit; BQL: Below Quantifiable Limit. [symbol]
indicates data missing or illegible when filed

The above stability results show that the pharmaceutical composition prepared in Example 4B is stable for at least six months.

TABLE 2
Dissolution testing and Stability study results of the
lumateperone compositions prepared in Example 5B.

| | | Storage condition (25° C./60% RH) | | |
|---|---|---|---|---|
| Sr. No. | Attributes | Initial | 3 Months | 6 Months |
| 1 | Assay (%) | 97.9 | 100.1 | 99.4 |
| 2 | Water content (%) | 0.2 | 0.2 | 0.2 |
| 3 | Dissolution (%) | 96 | 97 | 101 |
| | (0.1N HCl, 500 ml, | | | |
| | Paddle, 50 rpm, | | | |
| | with [symbol] | | | |
| 4 | Organic impurities (%) | | | |
| 4.1 | LMT1 impurities | Below LOQ | Below LOQ | Below LOQ |
| 4.2 | Cyclopropane | ND | ND | ND |
| | impurity | | | |
| 4.3 | Desfluoro impurity | Below LOQ | Below LOQ | Below LOQ |
| 4.4 | Dimer impurity | Below LOQ | Below LOQ | Below LOQ |
| 4.5 | Any unspecified | 0.02 | 0.03 | 0.07 |
| | degradation | | | |
| | product [symbol] | | | |
| 4.6 | Total degradation | 0.1 | 0.2 | 0.3 |

[symbol]

indicates data missing or illegible when filed

The above stability results show that the pharmaceutical composition prepared in Example 5B is stable for at least six months.

TABLE 3
Dissolution testing and Stability study results of the
lumateperone compositions prepared in Example 6B.

| | | Storage condition (25° C./60% RH) | | |
|---|---|---|---|---|
| Sr. No. | Attributes | Initial | 3 Months | 6 Months |
| 1 | Assay (%) | 97.9 | 100.1 | 99.4 |
| 2 | Water content (%) | 0.2 | 0.2 | 0.2 |
| 3 | Dissolution (%) | 96 | 97 | 101 |
| | (0.1N HCl, 500 ml, | | | |
| | Paddle, 50 rpm, | | | |
| | with [symbol] | | | |
| 4 | Organic impurities (%) | | | |
| 4.1 | LMT1 impurities | Below LOQ | Below LOQ | Below LOQ |
| 4.2 | Cyclopropane | ND | ND | ND |
| | impurity | | | |
| 4.3 | Desfluoro impurity | Below LOQ | Below LOQ | Below LOQ |
| 4.4 | Dimer impurity | Below LOQ | Below LOQ | Below LOQ |
| 4.5 | Any unspecified | 0.02 | 0.03 | 0.07 |
| | degradation | | | |
| | product [symbol] | | | |
| 4.6 | Total degradation | 0.1 | 0.2 | 0.3 |

[symbol]

indicates data missing or illegible when filed

The above stability results show that the pharmaceutical composition prepared in Example 6B is stable for at least six months.

### Example 9—Evaluation of Nitrosamine Impurities

As per FDA release guidance on Control of Nitrosamine Impurities in Human Drugs, Lumateperone Capsules 10.5 mg, 21 mg, and 42 mg, as prepared in Examples 4B, 5B, and 6B were evaluated as follows:
Phase-I (Risk Assessment of Raw Materials). The risk of N-Nitrosamine impurities with use of the raw materials used in the formulation including the packaging materials.
Phase-II (Risk Assessment of Manufacturing Process). The risk of N-Nitrosamine impurities contributing drug product from the equipment involved in the manufacturing process of the drug product.

### Example 9—Risk Assessment of API, Excipients, Packaging Material, and Equipment

| Variables | CQA | Justification |
|---|---|---|
| API | NDMA, NDIPA and | These impurities are theoretically possible. |
| | NIPEA | |
| | NDEA | This impurity is theoretically possible. |
| | NMBA, NDBA and | These impurities are not possible. |
| | NMPA | |
| Excipients | N-Nitroso | Based on raw material vendor's nitrosamine |
| | Lumateperone | assessments and declarations, the excipients |
| | | are free from possible Nitrosamine |
| | | impurities. |
| | LMT-1 Nitroso | Based on raw material vendor's nitrosamine |
| | impurity | assessments and declarations, the excipients |
| | | are free from possible Nitrosamine |
| | | impurities. |
| | NDMA, NDIPA and | Based on raw material vendor's nitrosamine |
| | NIPEA | assessments and declarations, the excipients |
| | | are free from possible Nitrosamine |
| | | impurities. |
| | NDEA | NDEA is possible impurity in sodium |
| | | stearyl fumarate. However, the use level of |
| | | sodium stearyl fumarate in proposed |
| | | composition is 2% w/w which is very low. |
| | NMBA, NDBA and | Based on raw material vendor's nitrosamine |
| | NMPA | assessments and declarations, the excipients |
| | | are free from possible Nitrosamine |
| | | impurities. |
| Packaging Materials | N-Nitroso | Based on the vendor's nitrosamine |
| | Lumateperone | assessments and declarations, the packaging |
| | | materials are free from possible |
| | | Nitrosamine impurities. |
| | LMT-1 Nitroso | Based on the vendor's nitrosamine |
| | impurity | assessments and declarations, the packaging |
| | | materials are free from possible |
| | | Nitrosamine impurities. |
| | NDMA, NDIPA and | Based on the vendor's nitrosamine |
| | NIPEA | assessments and declarations, the packaging |
| | | materials are free from possible |
| | | Nitrosamine impurities. |
| | NDEA | Based on the vendor's nitrosamine |
| | | assessments and declarations, the packaging |
| | | materials are free from possible |
| | | Nitrosamine impurities. |
| | NMBA, NDBA and | Based on the vendor's nitrosamine |
| | NMPA | assessments and declarations, the packaging |
| | | materials are free from possible |
| | | Nitrosamine impurities. |
| Manufacturing | N-Nitroso | The proposed generic product |
| Process and | Lumateperone | (Lumateperone Capsules 10.5 mg, 21 mg, |
| Equipment | LMT-1 Nitroso | and 42 mg) is manufactured using standard |
| | impurity | manufacturing process and the process is |
| | NDMA, NDIPA and | validated. The manufacturing process is |
| | NIPEA | simple which involves sifting, dry |
| | NDEA | granulation by roller compaction, blending, |
| | NMBA, NDBA and | lubrication, encapsulation and packaging |
| | NMPA | and it is very likely affect the nitrosamine |
| | | contents of the drug product. All the |
| | | Equipment's used for manufacturing are |
| | | made of stainless steel-316 which is non- |
| | | reactive and corrosion resistance. All the |
| | | Equipment's used in manufacturing process |
| | | are qualified as per cGMP Standard and |
| | | procedure. Thus, there is no risk of any |
| | | contribution of Nitrosamine impurities from |
| | | the manufacturing process and equipments |
| | | used in the manufacturing the of the generic |
| | | drug product. |

Example 10—Nitrosamine Impurity Study of Lumateperone Capsules Prepared in Examples 4B. 5B. and 6B. Three packaging configurations (30's HDPE, 100's HDPE, and Alu-Alu Blister) were used.

### Lumateperone Capsules 10.5 mg Prepared in Example 6B

| | | | | |
|---|---|---|---|---|
| Capsules | | Example 6B | Example 6B | Example 6B |
| Packaging | | 30's HDPE Bottle | 100's HDPE Bottle | 10's Alu-Alu Blister |
| Nitrosamine | Specification | Init/6 month/6 month | Init/6 month/6 month | Init/6 month/6 month |
| Impurities | | 25° C./60% RH/ | 25° C./60% RH/ | 25° C./60% RH/ |
| | | 40° C./75% RH | 40° C./75% RH | 40° C./75% RH |
| N-Nitroso | NMT 35.71 | BDL/BDL/BDL | BDL/BDL/BDL | BDL/BDL/3.51 |
| Lumateperone | ppm | | | |
| NDEA | NMT 0.63 | ND/ND/ND | ND/ND/ND | ND/ND/ND |
| | ppm | | | |
| NDMA | NMT 2.29 | ND/ND/ND | ND/ND/ND | ND/ND/ND |
| | ppm | | | |
| NDIPA | NMT 0.63 | ND/ND/ND | ND/ND/ND | ND/ND/ND |
| | ppm | | | |
| NIPEA | NMT 0.63 | ND/ND/ND | ND/ND/ND | ND/ND/ND |
| | ppm | | | |

The above stability results for Lumateperone Capsules 10.5 mg prepared in Example 6B demonstrated that there is no significant increase in the nitrosamine impurities when the drug product is stored at long term stability conditions. There is increase in the N-Nitroso Lumateperone impurity when the drug product is stored at accelerated, however, the value 3.51 is within the 10% of the acceptable limits of NMT 35.71 ppm (3.57 ppm).

### Lumateperone Capsules 21 mg Prepared in Example 5B

| | | | | |
|---|---|---|---|---|
| Capsules | | Example 5B | Example 5B | Example 5B |
| Packaging | | 30's HDPE Bottle | 100's HDPE Bottle | 10's Alu-Alu Blister |
| Nitrosamine | Specification | Init/6 month/6 month | Init/6 month/6 month | Init/6 month/6 month |
| Impurities | | 25° C./60% RH/ | 25° C./60% RH/ | 25° C./60% RH/ |
| | | 40° C./75% RH | 40° C./75% RH | 40° C./75% RH |
| N-Nitroso | NMT 35.71 | BDL/BDL/BDL | BDL/BDL/3.24 | BDL/BDL/BDL |
| Lumateperone | ppm | | | |
| NDEA | NMT 0.63 | ND/ND/ND | ND/ND/ND | ND/ND/ND |
| | ppm | | | |
| NDMA | NMT 2.29 | ND/ND/ND | ND/ND/ND | ND/ND/ND |
| | ppm | | | |
| NDIPA | NMT 0.63 | ND/ND/ND | ND/ND/ND | ND/ND/ND |
| | ppm | | | |
| NIPEA | NMT 0.63 | ND/ND/ND | ND/ND/ND | ND/ND/ND |
| | ppm | | | |

The above stability results for Lumateperone Capsules 21 mg prepared in Example 5B demonstrated that, there is no significant increase in the nitrosamine impurities when the drug product is stored at long term stability conditions. There is increase in the N-Nitroso Lumateperone impurity when the drug product is stored at accelerated, however, the value 3.24 is within the 10% of the acceptable limits of NMT 35.71 ppm (3.57 ppm)

### Lumateperone Capsules 42 mg Prepared in Example 4B

| | | | | |
|---|---|---|---|---|
| Capsules | | Example 4B | Example 4B | Example 4B |
| Packaging | | 30's HDPE Bottle | 100's HDPE Bottle | 10's Alu-Alu Blister |
| Nitrosamine | Specification | Init/6 month/6 month | Init/6 month/6 month | Init/6 month/6 month |
| Impurities | | 25° C./60% RH/ | 25° C./60% RH/ | 25° C./60% RH/ |
| | | 40° C./75% RH | 40° C./75% RH | 40° C./75% RH |
| N-Nitroso | NMT 35.71 | BDL/BDL/BDL | BDL/BDL/3.21 | BDL/BDL/BDL |
| Lumateperone | ppm | | | |
| NDEA | NMT 0.63 | ND/ND/ND | ND/ND/ND | ND/ND/ND |
| | ppm | | | |
| NDMA | NMT 2.29 | ND/ND/ND | ND/ND/ND | ND/ND/ND |
| | ppm | | | |
| NDIPA | NMT 0.63 | ND/ND/ND | ND/ND/ND | ND/ND/ND |
| | ppm | | | |
| NIPEA | NMT 0.63 | ND/ND/ND | ND/ND/ND | ND/ND/ND |
| | ppm | | | |

The above stability results for Lumateperone Capsules 42 mg prepared in Example 4B demonstrated that, there is no significant increase in the nitrosamine impurities when the drug product is stored at long term stability conditions. There is increase in the N-Nitroso Lumateperone impurity when the drug product is stored at accelerated, however, the value 3.21 is within the 10% of the acceptable limits of NMT 35.71 ppm (3.57 ppm). In contrast, Lumateperone Capsules 42 mg which were prepared by a wet granulation process and stored in HDPE bottle without dessicant/canister resulted in greater than 10% increase in the amount of nitrosamine impurities. Thus, the results in Example 10 clearly show that there is no significant increase in the nitrosamine impurities in the lumateperone compositions prepared in Examples 4B, 5B, and 6B which were exposed to one or more desiccants while being stored in closed 30's HDPE Bottle, 100's HDPE Bottle, or 10's Alu-Alu Blister for a period of six months at either 25° C./60% RH or 40° C./75% RH. Although the inventions herein have been described with reference to particular embodiments, it is to be understood that these embodiments are merely illustrative of the principles and application of the present invention. It is therefore to be understood that numerous modifications may be made to the illustrative embodiments and that other arrangements may be devised without departing from the spirit and scope of the present invention as described.

All publications, patents, and patent applications cited in this application are herein incorporated by reference to the same extent as if each individual publication, patent, or patent application was specifically and individually indicated to be incorporated herein by reference.

What is claimed is:

1. A pharmaceutical composition for oral administration comprising: a) about 5% w/w to about 40% w/w of lumateperone or pharmaceutically acceptable salt thereof; b) about 50% w/w to about 95% w/w of at least one diluent; c) about 1% w/w to about 10% w/w of at least one disintegrant; d) about 0.1% w/w to about 2% w/w of at least one glidant; and e) about 0.5% w/w to about 5% w/w of a lubricant selected from the group consisting of sodium stearyl fumarate, stearic acid, and combinations thereof, wherein the amount of nitrosamine impurity after exposure of the pharmaceutical composition to 40° C./75% RH for a period of six months is less than the FDA acceptable intake limit of the nitrosamine impurity based on maximum daily dose of lumateperone.

2. The pharmaceutical composition of claim 1 wherein the diluent is mannitol, the disintegrant is croscarmellose sodium, and the glidant is colloidal silicon dioxide.

3. The pharmaceutical composition of claim 2 wherein lumateperone or pharmaceutically acceptable salt thereof is present in an amount of about 10% w/w to about 30% w/w, mannitol is present in an amount of about 60% w/w to about 90% w/w, croscarmellose sodium is present in an amount of about 0.5% w/w to about 10% w/w, colloidal silicon dioxide is present in an amount of about 0.5% w/w to about 5% w/w, and sodium stearyl fumarate is present in an amount of about 0.5% w/w to about 5% w/w.

4. The pharmaceutical composition of claim 2, wherein lumateperone or pharmaceutically acceptable salt thereof is present in an amount of about 20% w/w; mannitol is present in an amount of about 74% w/w; croscarmellose sodium is present in an amount of about 2.7% w/w; colloidal silicon dioxide in present in an amount of about 1% w/w; and sodium stearyl fumarate is present in an amount of about 2% w/w.

5. The pharmaceutical composition of claim 2, wherein lumateperone or pharmaceutically acceptable salt thereof is present in an amount of about 5 to about 70 mg; mannitol is present in an amount of about 25 to about 250 mg; croscarmellose sodium is present in an amount of about 1 to about 25 mg; colloidal silicon dioxide in present in an amount of about 0.1 to about 5 mg; and sodium stearyl fumarate is present in an amount of about 0.5 to about 8 mg.

6. The pharmaceutical composition of claim 5, wherein lumateperone is present as the mono-tosylate salt in an amount of about 60 mg equivalent to about 42 mg of lumateperone; mannitol is present in an amount of about 223 mg; croscarmellose sodium is present in an amount of about 8 mg; colloidal silicon dioxide in present in an amount of about 3 mg; and sodium stearyl fumarate is present in an amount of about 6 mg.

7. The pharmaceutical composition of claim 6, wherein lumateperone is present as the mono-tosylate salt in an amount of about 30 mg equivalent to about 21 mg of lumateperone; mannitol is present in an amount of about 111 mg; croscarmellose sodium is present in an amount of about 4 mg; colloidal silicon dioxide in present in an amount of about 1.5 mg; and sodium stearyl fumarate is present in an amount of about 3 mg.

8. The pharmaceutical composition of claim 7, wherein lumateperone is present as the mono-tosylate salt in an amount of about 15 mg equivalent to about 10.5 mg of lumateperone; mannitol is present in an amount of about 56 mg; croscarmellose sodium is present in an amount of about 2 mg; colloidal silicon dioxide in present in an amount of about 0.75 mg; and sodium stearyl fumarate is present in an amount of about 1.5 mg.

9. A pharmaceutical composition for oral administration comprising: a) about 5% w/w to about 40% w/w of lumateperone or pharmaceutically acceptable salt thereof; b) about 50% w/w to about 95% w/w of mannitol; c) about 1% w/w to about 10% w/w of croscarmellose sodium; d) about 0.1% w/w to about 2% w/w of colloidal silicon dioxide; and e) about 0.5% w/w to about 5% w/w of sodium stearyl fumarate; wherein the mannitol, colloidal silicon dioxide, and sodium stearyl fumarate are present as intragranular excipients, and the croscarmellose sodium, colloidal silicon dioxide, and sodium stearyl fumarate are present as extragranular excipients, wherein the total amount of the intragranular and extragranular excipients are within said weight percents.

10. The pharmaceutical composition of claim 9, wherein lumateperone is present as the mono-tosylate salt in an amount of about 15 mg equivalent to about 10.5 mg of lumateperone; mannitol is present in an amount of about 56 mg; croscarmellose sodium is present in an amount of about 2 mg; colloidal silicon dioxide in present in an amount of about 0.75 mg; and sodium stearyl fumarate is present in an amount of about 1.5 mg.

11. The pharmaceutical composition of claim 9, wherein about 50% of the sodium stearyl fumarate is present as an intragranular excipient and 50% of the sodium stearyl fumarate is present as an extragranular excipient.

12. A pharmaceutical composition for oral administration comprising an intragranular portion and an extragranular portion, wherein the intragranular portion comprises lumateperone or pharmaceutically acceptable salt thereof, mannitol, croscarmellose sodium, colloidal silicon dioxide; and sodium stearyl fumarate; and the extragranular portion comprises croscarmellose sodium, colloidal silicon dioxide, and sodium stearyl fumarate, wherein at least part of the colloidal silicon dioxide and sodium stearyl fumarate are present in the intragranular portion and at least part of the colloidal silicon dioxide and sodium stearyl fumarate are present in the extragranular portion.

13. The pharmaceutical composition of claim 10 wherein the lumateperone mono-tosylate is present in an amount equivalent to 0.01 to 30 mg of lumateperone free base.

14. The pharmaceutical composition of claim 10, wherein the lumateperone mono-tosylate salt is present in the form of particles having a particle size D90 of 10 μm to 150 μm.

15. The pharmaceutical composition of claim 14, wherein the lumateperone mono-tosylate salt is present in the form of particles having a particle size D90 of 70 μm to 100 μm.

16. The pharmaceutical compositions of claim 1, which is in the form of a capsule wherein a single capsule dissolves in 500 mL of 0.1N aqueous hydrochloric acid to the extent of at least 85% after 15 minutes, and/or to the extent of at least 92% after 30 minutes, and/or at least 94% after 45 minutes.

17. A process for preparing the pharmaceutical composition of claim 1, wherein the process comprises the steps of: (a) mixing lumateperone or a pharmaceutically acceptable salt thereof, and at least one diluent, at least one glidant, and at least one lubricant to form a mixture; (b) granulating the mixture from Step (a) to form granules; (c) mixing at least one disintegrant, at least one lubricant, and at least one glidant with the granules formed in Step (b) to form a lubricated blend; and (d) encapsulating or tableting the lubricated blend from Step (c).

18. A process for preparing the pharmaceutical composition of claim 1, wherein the process comprises the steps of: (i) mixing lumateperone or a pharmaceutically acceptable salt thereof, and at least one diluent, to form a mixture; (ii) adding at least one glidant and at least one lubricant to the mixture from Step (i), to form a blend of particles; (iii) mixing at least one disintegrant, at least one lubricant, and at least one glidant with the blend of particles from Step (ii), to form a lubricated blend of particles; and (iv) encapsulating or tableting the lubricated blend of particles from Step (iii).

19. A process for preparing the pharmaceutical composition of claim 1, wherein the process comprises the steps of: (I) mixing lumateperone or a pharmaceutically acceptable salt thereof, and at least one diluent, to form a mixture; (II) adding at least one glidant to the mixture from Step (I), to form a combination of particles; (III) sifting the combination of particles from Step (II) to form a sifted mixture of particles; (IV) milling the sifted mixture of particles from Step (III), to form milled particles; (V) mixing the milled particles from Step (IV) with at least one disintegrant, at least one lubricant, and at least one glidant, to form a lubricated blend of particles; and (VI) encapsulating or tableting the lubricated blend of particles from Step (V).

20. A process for preparing the pharmaceutical composition of claim 1, wherein the process comprises the steps of: (A) preparing a mixture of lumateperone or a pharmaceutically acceptable salt thereof, at least one diluent, at least one glidant, and at least one lubricant; (B) granulating the mixture of Step (A) to form intragranular particles; (C) mixing at least one disintegrant, at least one lubricant, and at least one glidant with the intragranular particles from Step (B) to form a mixture of intragranular particles and extragranular particles; and (D) encapsulating or tableting the mixture of intragranular and extragranular particles from Step (C).
